(19) United States
(12) Patent Application Publication
(10) Pub. No.: US 2006/0205973 A1
(43) Pub. Date: Sep. 14, 2006

(54) Process for preparing alkyl esters of (meth)acrylic acid

(75) Inventors: Thorsten Habeck, Meckenheim (DE); Ulrich Rauh, Frankenthal (DE); Thorsten Friese, Mannheim (DE); Hans Martan, Frankenthal (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/358,220

(22) Filed: Feb. 22, 2006

(30) Foreign Application Priority Data
Mar. 8, 2005 (DE) 102005010587.4

(51) Int. Cl. C07C 67/36

(52) U.S. Cl. 560/206

(57) ABSTRACT

A process is described for continuously preparing alkyl esters of (meth)acrylic acid by reacting (meth)acrylic acid and alkanols having from 1 to 5 carbon atoms in homogeneous, liquid, solvent-free phase at elevated temperature and in the presence of an acidic esterification catalyst, in which
the (meth)acrylic acid, the alkanol and the acidic esterification catalyst are fed to a reaction zone (5, 6),
a reaction mixture (21) is discharged from the reaction zone (5, 6), and is introduced into a rectification unit I and separated by addition of water into a top stream (23), comprising the alkyl ester of (meth)acrylic acid, and a bottom stream (22), and
a substream of the bottom stream (22) from the rectification unit I is fed to a residue dissociation unit IV, which comprises a dissociating boiler (61), and is separated therein into a top stream (29), which is recycled into the rectification unit I and a bottom stream (40), which is removed from the system,
wherein the residue dissociation unit IV, as well as the dissociating boiler (61), further comprises a rectification column (62), the water content of the liquid obtained at the bottommost stage thereof being less than 0.1% by weight.

The invention relates to a process for continuously preparing alkyl esters of (meth)acrylic acid by reacting (meth)acrylic acid and alkanols having from 1 to 5 carbon atoms in homogeneous liquid solvent-free phase at elevated temperature and in the presence of an acidic esterification catalyst.

In the present context, the term (meth)acrylic acid refers in a known manner to acrylic acid and/or methacrylic acid.

Alkyl esters of (meth)acrylic acid are well known and are of significance, for example, as starting monomers for the preparation of aqueous polymer dispersions which find use, for example, as adhesives.

Processes for preparing alkyl esters of (meth)acrylic acid by reacting (meth)acrylic acid with monohydric alkanols having from 1 to 5 carbon atoms in homogeneous liquid phase, at elevated temperature and in the presence of proton-supplying catalysts are known and described, for example, in DE-A 14 68 932, 22 26 829 and 22 52 334. These are typical equilibrium reactions in which the degree of conversion of (meth)acrylic acid and of the particular alkanol to the corresponding ester is restricted significantly by the equilibrium constant. This has the consequence that, for an economically viable process, the unconverted starting materials have to be removed from the ester formed and recycled into the reaction zone. It is generally found to be particularly difficult to remove the ester formed from unconverted (meth)acrylic acid, since their boiling points are usually comparatively close together. Various measures for increasing the conversion of (meth)acrylic acid to the corresponding esters have therefore already been proposed, for example the use of an increased molar excess of alkanol compared to the (meth)acrylic acid, the removal of the water of reaction by means of an organic entraining agent which forms a suitable azeotrope, or the extraction of the ester formed with a suitable solvent during the reaction. However, these processes have the disadvantage that a large excess of alkanol has to be recovered or the entraining agent or the extractant has to be isolated. An increased alkanol excess additionally increases the formation of its dialkyl ether as a by-product.

GB-1017522 discloses a process for preparing n-butyl acrylate. The esterification conditions recommended by GB-1017522 are a molar ratio of starting alkanol to starting acid of from 2.3 to 5, and a content, based on the total mass of the reactants, of catalytically active sulfuric acid or organic sulfonic acid of from 0.5 to 5% by weight. A disadvantage of this procedure is the required increased excess of starting alkanol which promotes the formation of undesired dialkyl ether, and also the yield, which is not fully satisfactory under the aforementioned conditions, of n-butyl acrylate based on the amount of acrylic acid used.

DE-B 25 52 987 discloses a process for continuously preparing alkyl esters of acrylic acid by reaction of acrylic acid and monohydric alkanols having from 1 to 4 carbon atoms in homogeneous, liquid, solvent-free phase in a molar ratio of from 1 (alkanol):1 (acrylic acid) to 2 (alkanol):1 (acrylic acid) at elevated temperature and in the presence of sulfuric acid or organic sulfonic acid as a catalyst, in which the acrylic acid, the alkanol and the acid catalyst are fed continuously to a reaction zone, the alkyl acrylate formed is removed rectificatively over a residence time of several hours as a constituent of at least one aqueous azeotrope consisting not only of the alkyl acrylate but also of water or water and starting alcohol as a further constituent via the top of a rectification column attached to the reaction zone and having a top pressure of from 0.1 to 1 atm, the distillate I obtained is separated into an organic phase which comprises the acrylic ester formed and into an aqueous phase, a portion of the organic phase is recycled via the top of the rectification zone for the purposes of generating increased separating action, as is, if appropriate, a portion of the aqueous phase to maintain the composition of the aqueous azeotrope, the alkyl ester is removed in a manner known per se from the excess organic phase, and a portion of the reaction mixture is discharged from the reaction zone and freed of high boilers by distillation, and the distillate II obtained is recycled into the reaction zone.

The primary objective of DE-B 25 52 987 is the prevention of undesired ether formation from starting alkanol. However, a disadvantage of the procedure of DE-B 25 52 987 is that, in spite of distillative treatment of the effluent from the reaction mixture and recycling of the distillate obtained into the reaction zone, the yield of alkyl acrylate based on acrylic acid used is not satisfactory. Nor is the achieved reduction in the dialkyl ether by-product formation fully satisfactory. Furthermore, the residence time required in the working examples is not satisfactory. This is also true of the space-time yield. It is assumed that this results from the low concentration of acidic esterification catalyst.

It has therefore already been proposed (EP-A 0 733 617) to carry out the corresponding esterification process in the presence of increased concentrations of an acidic esterification catalyst, which promotes the dissociation of oxy esters formed as further by-products in the esterification and thus increases the yield of ester based on (meth)acrylic acid used at a given residence time.

Furthermore, it has already been proposed (DE-A 195 36 178) at a simultaneously high yield of ester, to achieve a further reduction in the amount of dialkyl ether by the reaction zone consisting of a battery of at least two reaction regions connected in series and preferably operated continuously, and the liquid effluent stream of one reaction region forming the feedstream of the downstream reaction region.

DE-A 196 04 252 proposes an improved process for preparing alkyl esters of (meth)acrylic acid in which the alkyl ester of (meth)acrylic acid formed is removed as a constituent of an azeotrope with water from the reaction mixture of the esterification via the top of the rectification column, and the acid esterification catalyst and the (meth)acrylic acid reactant are removed via the bottom.

It was accordingly an object of the invention to provide an improved process for preparing alkyl esters of (meth)acrylic acid, with the alkyl ester of (meth)acrylic acid formed being removed as a constituent of an azeotrope with water, in which the corrosion potential in the dissociating boiler, which is used to remove high boilers from the bottom liquid of the azeotropic column, is significantly reduced.

This object is achieved by a process for continuously preparing alkyl esters of (meth)acrylic acid by reacting (meth)acrylic acid and alkanols having from 1 to 5 carbon atoms in homogeneous, liquid, solvent-free phase at elevated temperature and in the presence of an acidic esterification catalyst, in which
the (meth)acrylic acid, the alkanol and the acidic esterification catalyst are fed to a reaction zone (5, 6),
the water formed during a residence time is removed rectificatively as a constituent of an alkanol-comprising mixture in a rectification unit III attached to the reaction zone (5, 6),
the distillate obtained here is separated into an alkanol-comprising organic phase and a water-comprising aqueous phase, the organic phase is recycled into the rectification unit III and the aqueous phase is discharged, and a reaction mixture (21) being discharged from the reaction zone (5, 6), and
is introduced into a rectification unit I and separated by addition of water into a top stream (23), comprising the alkyl ester of (meth)acrylic acid, and a bottom stream (22), and
a substream of the bottom stream (22) from the rectification unit I is fed to a residue dissociation unit IV, which comprises a dissociating boiler (61), and is separated therein into a top stream (29), which is recycled into the rectification unit I and a bottom stream (40), which is removed from the system,
   wherein the residue dissociation unit IV, as well as the dissociating boiler (61), further comprises a rectification column (62), the vapor ascending from the dissociating boiler (61) being introduced into the rectification column (62) and the liquid which is obtained at the bottommost stage of the rectification column (62) is routed from the rectification column (62) into the dissociating boiler (61), and the rectification column (62) being operated such that the water content of the liquid obtained at the bottommost stage thereof is less than 0.1% by weight.

The process stage of esterification may be performed in any known embodiment in which alkyl esters of (meth)acrylic acid are obtained continuously by reacting (meth)acrylic acid and alkanols having from 1 to 5 carbon atoms in homogeneous liquid solvent-free phase at elevated temperature and in the presence of an acidic esterification catalyst, in which the (meth)acrylic acid, the alkanol and the acidic esterification catalyst are fed to a reaction zone, the water formed during a residence time is removed rectificatively as a constituent of an alkanol-comprising mixture in a rectification unit attached to the reaction zone, the distillate obtained here is separated into an alkanol-comprising organic phase and a water-comprising aqueous phase, the organic phase is recycled into the rectification unit and the aqueous phase is discharged.

Such a process is described, for example, in DE-A 196 04 252.

According to this, the esterification is operated advantageously under reduced pressure to remove the water of reaction and is separated from the removal of the alkyl(meth)acrylate which follows in spatial terms and also in control technology terms. Esterification and subsequent removal of the alkyl(meth)acrylate in the rectification zone are therefore very flexibly adjustable. Water which is passed into the second rectification zone for the azeotropic removal of the alkyl(meth)acrylate therefore influences the esterification only slightly.

The reaction zone consists of one or more reaction regions. In the embodiment of the invention with a plurality of reaction regions, it is advantageous to arrange them in a battery. In that case, the liquid effluent stream of one reaction region forms the feed of the downstream reaction region. This can be done with the aid of an overflow. In the case that the individual reaction regions are apparatuses separated from one another, their number, taking into account the capital costs, is >=2 and <=4. When more than one reaction region is provided within one and the same reactor (for example by the use of dividing sheets), the number of reaction regions may also be greater than 4. In the case of a plurality of reaction regions, the vapors of the reaction regions are fed to a combined rectification column whose liquid effluent advantageously passes into the first reaction region. After the condensation, the distillate is divided into two phases, an organic phase consisting substantially of starting alkanol, and an aqueous phase consisting substantially of water, and the organic phase is passed substantially fully, more preferably fully, back to the rectification unit III.

The temperature of the reaction mixture in the different reaction regions corresponds normally to the boiling temperature of the particular reaction mixture at the pressure established, preferably from 0.1 to 1 atm, more preferably from 0.1 to 0.5 atm. In other words, it normally increases along the battery (in the case of a plurality of reaction regions) toward the bottom of the rectification unit I.

The separation of esterification reaction and distillative removal of the alkyl ester of (meth)acrylic acid allows milder reaction conditions. The reaction can be executed in all reaction regions at a pressure of from 100 mbar to atmospheric pressure, preferably from 200 to 700 mbar, more preferably from 300 to 450 mbar top pressure (water removal column), and a temperature of from 90° C. to 115° C.

The pressure may be the same in all reaction regions. The rectification unit I is preferably operated at standard pressure and at ≧100° C. and ≦130° C. The temperature in the rectification units connected downstream of the reaction zone should not exceed 135° C. in order to suppress undesired polymerizations as side reactions.

In an advantageous embodiment of the invention, the content of catalytically active acid in the first reaction region, based on the reaction mixture comprised therein, is from 0.1 to 10% by weight, preferably between 0.1 and 6% by weight, of para-toluenesulfonic acid or of an equimolar amount of organic sulfonic acid and/or sulfuric acid. The total residence time of the reactants in the reaction zone is generally from 0.25 to 15 hours, preferably from 1 to 7 h, more preferably from 2 to 5 h. In the bottom of the rectification unit I, it is preferably from 0.2 to 5 hours.

According to the present invention, the residue dissociation unit IV is configured such that it, as well as the dissociating boiler, further comprises a rectification column which is operated such that the liquid which is obtained at the bottommost tray of the rectification column and is routed into the dissociating boiler has a much reduced water content, of less than 0.1% by weight, based on the total weight of the liquid stream. Preferably, the rectification column is operated such that the water content of the liquid from the bottommost tray thereof is less than 200 weight ppm, based on the total weight of the liquid stream.

By providing a rectification column which is operated as described above, acidic esterification catalyst and water will no longer continue to be present at one and the same time in the dissociating boiler; this distinctly reduces the corrosion problem in the dissociating boiler, so that this apparatus does not have to be made of costly, corrosion-resistant specialty alloys, as required in the prior art process, but that inexpensive materials of construction will do.

The dissociating boiler and the rectification column may be configured as a single apparatus; that is, the dissociating boiler may be integrated in the bottom region of the rectification column or constitute that bottom region of the rectification column.

In a further version of the process, the dissociating boiler and the rectification column are configured as spatially separated apparatuses.

The heat supply into the dissociating boiler can be via an internal vaporizer system and/or via an external heat transferrer.

The rectification column preferably comprises from two to 5 theoretical separating stages.

It is advantageous to improve the gas/liquid transition by providing separation-active internals in the rectification column, preferably trays, beds or packings, more preferably dual flow trays.

The rectification column is advantageously operated as a stripping column; that is, the feed stream is applied in the top region of the column and there is no need for a rectifying section.

Preference is given to an operating mode where the temperature in the dissociating boiler does not exceed 200° C., preferably 180° C., and the top pressure in the rectification column is in the range from 300 mbar to atmospheric pressure, preferably in the range between 600 and 900 mbar.

Advantageously, the vapor stream from the rectification column can be routed into a condenser, condensed and the condensate advantageously fully recycled into the rectification unit I without a substream thereof being introduced as reflux back into the rectification column.

For the purpose of discharge of high boilers, a portion of the bottoms liquid from the rectification unit I is fed, preferably continuously, to a residue dissociation unit IV in which the low boilers are removed from the high boilers (oligomers and polymers formed), preferably in one stage and batchwise. These low boilers are substantially alkyl(meth)acrylate, starting alkanol and (meth)acrylic acid. For the purpose of increasing the yield, they are recycled into the rectification unit I. In the residue dissociation unit IV, a portion of the oxy esters is likewise dissociated, so that the losses of product of value can be kept very low.

In order to restrict the fraction of high boiling by-products which cannot be dissociated, it is sufficient to discharge an amount of from 1 to 20% by weight, preferably from 2 to 10% by weight, based on the feed amount of reactants to the reaction zone, from the residue dissociation unit IV into the residue dissociation unit IV. The amount of high boilers excluded from this residue dissociation unit IV is from 3 to 30% by weight, generally from 5 to 15% by weight, based on the stream fed thereto. The total losses based on the alkyl acrylate product of value amount to less than 1.5%.

In accordance with the amount of catalyst comprised in the discharged amount of high boilers from the residue dissociation unit IV, fresh catalyst is supplemented, preferably continuously, into the first reaction region. This leads to a steady state of the required concentration of acidic esterification catalyst in the reaction zone. The circulation makes catalyst workup superfluous and reduces the need for fresh catalyst. Discharge also affects the process stabilizer, so that its content levels off at a steady-state value.

The rectification unit I in which the target ester is drawn off with addition of water as a constituent of an azeotrope can be operated as known from the prior art, in particular DE-A 196 04 252.

The top stream drawn off from the rectification unit I is preferably condensed in a condenser, the condensate is separated in a phase separator into an organic phase and an aqueous phase, and the aqueous phase is partly reintroduced as reflux to the rectification unit I and otherwise discharged, and the organic phase is fed to a further rectification unit II and separated therein into a sidestream comprising the pure alkyl ester of (meth)acrylic acid, a top stream comprising the alkanol, and a bottom stream.

The organic phase of the top stream of the rectification unit I comprises the target ester as the main component and additionally alkanol and water. (Meth)acrylic acid and alkoxyalkyl esters of (meth)acrylic acid do not get into the top product as a result of a suitable adjustment of the operating parameters in the rectification unit I and require no further removal.

The downstream rectification unit II (alkanol/target ester removal) is preferably operated in such a way that alkanol with small fractions of water and alkyl acrylate is withdrawn as the top product at the upper end thereof and is passed back into the reaction zone, and that pure target ester is withdrawn at the lower end.

A particularly preferred embodiment of the alkanol/target ester removal consists in withdrawing the pure ester at the lower end of the rectification column II above the evaporator, between evaporator and fifth tray, most suitably above the evaporator as a vaporous side draw. This gas stream is condensed and stabilized in a known manner by replacing the process stabilizer with storage stabilizers, for example hydroquinone monomethyl ether. To prevent accumulation of high boilers, a substream, generally between 1 and 20%, in particular between 1 and 5%, of the feed amount to the rectification unit II is withdrawn from the evaporator of the rectification unit II and recycled into the catalyst removal unit V and/or into the rectification unit I.

Advantageously, the top product of the rectification unit II is recycled into the upper section of the rectification column III attached to the reaction zone in order to prevent the water comprised therein from getting into the reaction mixture.

Stabilizer-containing solution is preferably introduced in each case via the top to the rectification units I, II and III. For this purpose, customary stabilizers (polymerization inhibitors) are used, generally, based on the amount of α,β-monoethylenically unsaturated monomers, from 0.01 to 0.1% by weight. Useful stabilizers are, for example, phenolic compounds such as hydroquinone, hydroquinone monomethyl ether, but also para-benzoquinone, phenothiazine, methylene blue and/or air.

Particular preference is given to using the process according to the invention to prepare n-butyl acrylate.

The vapors forming in the rectification unit I spatially separated from the reaction zone are, as already described, fed to a rectification zone. With regard to the target ester-comprising mixture removed overhead therefrom, it is possible to distinguish between essentially two configurations. When the mixture is a heteroazeotrope, as, for example, in the case of the preparation of n-butyl acrylate, the azeotrope separates after it has been condensed by itself into an aqueous phase and into an organic phase. The aqueous phase consists normally mainly of water and some alkanol; the organic phase consists generally substantially of the ester formed and alkanol. To adjust the rectificative separating action, an appropriate portion of the organic phase is recycled via the top of the rectification zone.

To maintain the composition of the aqueous azeotrope, an appropriate portion of the aqueous phase is recycled into the rectification zone I, preferably likewise via the top of the attached rectification column. Alkanol comprised can be removed from the unrecycled fraction of the aqueous phase, for example by stripping (for example with air or steam), and recycled into the reaction zone. Appropriately, the recycling is by a direct route. The substantially pure water obtained is discharged.

When the aqueous azeotrope which comprises the target ester and is removed continuously via the top of the rectification zone in the process according to the invention is not a heteroazeotrope, this azeotrope does not separate by itself after it has been condensed into an aqueous phase and into an organic phase. However, this separation can be achieved in a simple manner, for example, by extracting the alkanol comprised in the azeotrope by means of water and rectificatively separating the water/alkanol mixture obtained. The alkanol is appropriately recycled into the reaction zone, preferably via the top of the attached rectification zone.

In the presence of a heteroazeotrope, a particularly preferred embodiment consists in passing the excess aqueous phase (water of reaction from the esterification) obtained at the top of the rectification column III attached to the reaction zone to the top product of the rectification zone I. The aqueous phase of this heteroazeotrope absorbs less alcohol after the demixing of the phases owing to the high content of alkyl(meth)acrylate and the lower alkanol content in the organic phase. The excess water of reaction which comprises between 1% by weight and 5% by weight, on average 2.5% by weight, of alkanol may be discharged from this water phase obtained at the top of the rectification unit I. In general, stripping of the alkanol as a further process step can be dispensed with.

Typically, the azeotrope drawn off from the rectification unit I, when the rectificative separating action is adjusted correctly, comprises no starting acid. When, however, this is not the case, it is possible to extractively remove the starting acid by means of water or of an alkaline solution and to work up the extract, if appropriate, subsequently in a manner known per se.

The process according to the invention has the particular feature that the corrosion potential in the dissociating boiler of the residue dissociation unit IV is distinctly reduced compared to known processes. This apparatus can therefore be made from substantially less expensive materials and the capital costs for the process are reduced correspondingly. Existing plants can easily be retrofitted.

The invention is illustrated in detail below with reference to a drawing and to a working example.

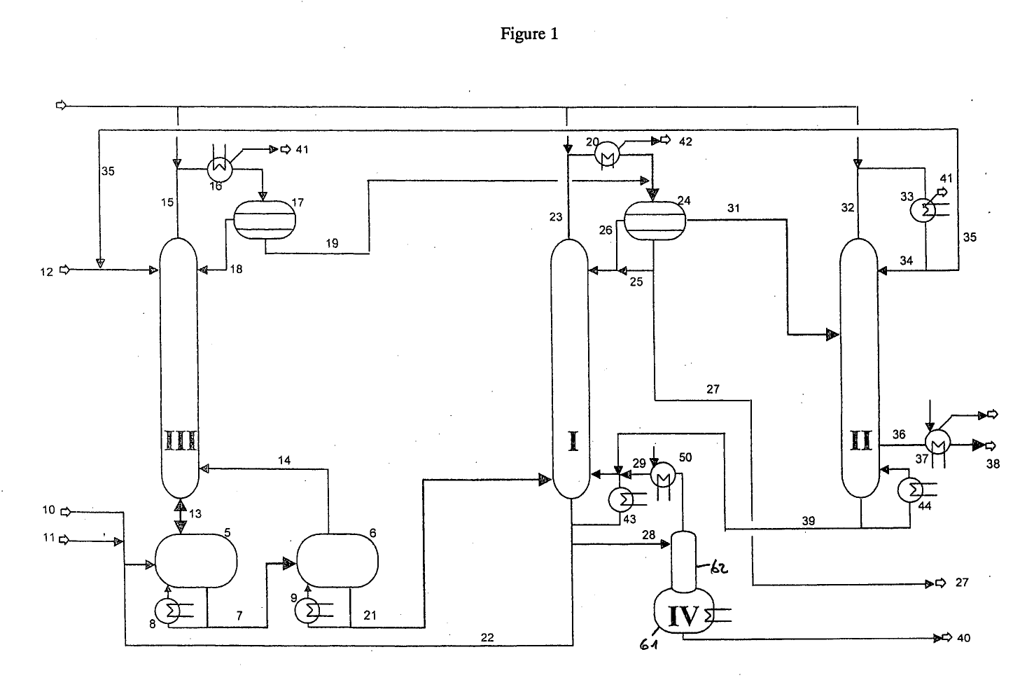

FIG. 1 shows the schematic illustration of a preferred embodiment of an inventive plant.

Roman numerals I to III indicate rectification units, and IV the residue dissociation unit.

Arabic numerals serve both to designate streams and to designate apparatuses.

The plant, shown in the drawing, for carrying out the inventive process for the preparation of n-butyl acrylate has three rectification columns I to III and a residue dissociation unit IV, which comprises a dissociating boiler 61 and a rectification column 62, the preferred embodiment depicted in the figure having the rectification column 62 sitting on top of the dissociating boiler 61. It is also equipped with two esterification reactors 5 and 6 which are connected in series via a line 7 and form a reaction battery. Circulation evaporators 8 and 9 are connected to the esterification reactors 5 and 6. 10 designates the feed line of acrylic acid, 11 that of para-toluenesulfonic acid and 12 the feed line of n-butanol.

The vapors ascending out of reactors 5 and 6 are introduced via lines 13 and 14 into the rectification unit III. The top product 15 from the rectification unit III is condensed in a condenser 16 and separated in a separator 17 into an organic phase 18 comprising butanol, butyl acrylate, water and butyl acetate, and an aqueous phase comprising butanol, butyl acrylate and butyl acetate. The organic phase 18 is recycled fully to the top of the rectification unit III.

The liquid effluent crude ester from the second esterification reactor 6 is fed via line 21 to the rectification unit I, in the lower region thereof.

The rectification stage I is equipped with a bottom evaporator 43 and a condenser 20 at the top of the column. The bottom stream 52 from the rectification unit I is recycled into the esterification stage, specifically the first esterification reactor 5. The top stream 23 is condensed in a condenser 22 at the top of the column, discharged partly as stream 42 and otherwise separated in a phase separator 24 into an aqueous stream 26 which is partly introduced back to the rectification stage I and otherwise discharged as stream 27. The organic phase 31 from the phase separator 24 is introduced into the rectification stage II which is likewise equipped with a bottom evaporator 44 and a condenser 33 at the top of the column. The top stream 32 from the rectification stage II is condensed in the condenser 33, discharged partly as stream 41 and otherwise introduced as reflux 34 back to the rectification stage II.

From the rectification stage II, a side stream 36 is drawn off, condensed in a condenser 37 and drawn off as pure target ester (stream 38).

A substream 28 of the bottom stream from the rectification unit I is introduced into the residue dissociation unit IV, namely in the preferred embodiment depicted in the figure in the upper region of the rectification column 62 which sits on the dissociating boiler 61. The vapor stream from the rectification column 62 is condensed in a condenser 50 and the condensate 29 is fully recycled into the rectification unit I, into the lower region thereof.

The bottom stream 40 from the dissociating boiler 61, comprising high boilers, is removed from the system.

Working Example

In a plant according to the schematic illustration in FIG. 1, a feed stream of 8.2 kg/h at 110° C. and 1.2 bar, comprising 7.9% by weight of water, 5.4% by weight of butanol, 39.8% by weight of n-butyl acrylate, 29.0% by weight of acrylic acid, 13.5% by weight of high boilers (in particular oxyesters), 3.3% by weight of paratoluenesulfonic acid and also traces of low boilers and stabilizer was fed onto the uppermost tray of the rectification column of a residue dissociation unit IV, comprising a dissociating boiler with internal heat transferrer and, sitting on top, a rectification column having an internal diameter of 0.05 m and 10 bubblecap trays and also a condenser at the top of the column. The top pressure of the rectification column was 800 mbar. The water content has decreased to 120 weight ppm in the still of the rectification column, i.e., the internal dissociation boiler, so that, despite the paratoluenesulfonic acid being concentrated to above 23% by weight, the dissociation of the strong acid and hence its corrosion potential was suppressed.

The dissociation or concentrating of the high boilers took place in the still, and they were removed from the system via the bottom stream.

The vapor from the rectification column was condensed in the condenser at the top of the column and stabilized. The condensate stream of 7.4 kg/h comprised 9.1% by weight of water, 3.1% by weight of butanol, 54% by weight of n-butyl acrylate and 32.6% by weight of acrylic acid.

What is claimed is:

1. A process for continuously preparing alkyl esters of (meth)acrylic acid by reacting (meth)acrylic acid and alkanols having from 1 to 5 carbon atoms in homogeneous, liquid, solvent-free phase at elevated temperature and in the presence of an acidic esterification catalyst, in which
the (meth)acrylic acid, the alkanol and the acidic esterification catalyst are fed to a reaction zone,
the water formed during a residence time is removed rectificatively as a constituent of an alkanol-comprising mixture in a rectification unit III attached to the reaction zone,
the distillate obtained here is separated into an alkanol-comprising organic phase and a water-comprising aqueous phase, the organic phase is recycled into the rectification unit III and the aqueous phase is discharged, and a reaction mixture being discharged from the reaction zone, and
is introduced into a rectification unit I and separated by addition of water into a top stream, comprising the alkyl ester of (meth)acrylic acid, and a bottom stream, and
a substream of the bottom stream from the rectification unit I is fed to a residue dissociation unit IV, which comprises a dissociating boiler, and is separated therein into a top stream, which is recycled into the rectification unit I and a bottom stream, which is removed from the system,
wherein the residue dissociation unit IV, as well as the dissociating boiler, further comprises a rectification column, the vapor ascending from the dissociating boiler being introduced into the rectification column and the liquid which is obtained at the bottommost stage of the rectification column is routed from the rectification column into the dissociating boiler, and the rectification column being operated such that the water content of the liquid obtained at the bottommost stage thereof is less than 0.1% by weight.

2. The process according to claim 1 wherein the water content of the liquid obtained at the bottommost stage of the rectification column is less than 200 weight ppm.

3. The process according to claim 1, wherein the alkyl ester of (meth)acrylic acid is n-butyl acrylate and/or the acidic esterification catalyst is paratoluenesulfonic acid.

4. The process according to claim 1, wherein the dissociating boiler is integrated in the rectification column.

5. The process according to claim 1, wherein the dissociating boiler and the rectification column are spatially separated.

6. The process according to claim 1, wherein the rectification column comprises from two to five theoretical separating stages.

7. The process according to claim 1, wherein the rectification column is configured as a stripping column, and the feed stream to the rectification column is introduced into the top region thereof.

8. The process according to claim 1, wherein the rectification column is equipped with separation-active internals.

9. The process according to claim 1, wherein the temperature in the dissociating boiler is less than 200° C. and the top pressure in the rectification column is between 300 mbar and atmospheric pressure, and preferably the temperature in the dissociating boiler is less than 180° C. and the top pressure in the rectification column is in the range from 600 to 900 mbar.

10. The process according to claim 1, wherein the vapor stream from the rectification column is routed into a condenser at the top of the rectification column and the condensate stream from the condenser is not applied as reflux at the top of the rectification column but is fully recycled into the rectification unit I.

11. The process according to claim 8, wherein the rectification column is equipped with trays, beds or packings.

12. The process according to claim 11, wherein the trays are dual flow trays.
